(19) United States
(12) Patent Application Publication
(10) Pub. No.: US 2025/0107706 A1
(43) Pub. Date: Apr. 3, 2025

(54) EYE EXAMINATION DEVICE

(71) Applicant: Shuji YOKOYAMA, Hokkaido (JP)

(72) Inventor: Shuji YOKOYAMA, Hokkaido (JP)

(21) Appl. No.: 18/684,727

(22) PCT Filed: Aug. 15, 2022
(86) PCT No.: PCT/JP2022/030866

§ 371 Date: Feb. 19, 2024

(30) Foreign Application Priority Data
Aug. 19, 2021 (JP) 2021-133742

(51) Int. Cl. A61B 3/032

(52) U.S. Cl. CPC A61B 3/032

(57) ABSTRACT

An object of the present invention is to provide a visual acuity test apparatus capable of displaying all test symbols provided for each test value by one test apparatus without using a visual acuity chart in which Landolt rings and characters of each country arranged in order according to a test value are displayed on a paper medium or an electric bulletin board, a test symbol card for optometry carried in hand, a precision machine or the like in combination.

A means for solving the above-described problem is to equip a visual acuity test apparatus with various types of test symbols by computer control, to enable the test symbols to be displayed in a stepless size by enlarging or reducing the test symbols by comparison in accordance with the test value according to the standards indicated by the Japanese Standards Association, and to change each of the test symbols individually and completely randomly even in a case where a plurality of test symbols is to be displayed at a time, or to optionally select to display the test symbol by an examiner.

### TECHNICAL FIELD

The present invention relates to a visual acuity test apparatus capable of steplessly selecting a visual acuity test value, and capable of supporting any subject without limiting a type, a size, and a direction of a test symbol by randomly or optionally selecting and displaying the test symbol for all test values.

### BACKGROUND ART

Regarding Patent Literature 1, claim 1 in claims recites a test method using a storage means that stores patterns of Landolt rings supporting a plurality of visual acuities, and the paragraph describes that “(omitted) note that, a pattern of a Landolt ring 30 displayed each time is randomly extracted from patterns stored in a memory 10”; it is disclosed that the pattern is randomly extracted from the provided patterns of the Landolt rings, and movement of the Landolt rings is not random, so that with human memory, a test symbol to be displayed next is stored after several times of optometry. The paragraph describes that a character and the Landolt ring can be used in combination, or symbols and figures other than them can be used regarding the type of test symbol, but there is no specific description of how to demonstrate the combination with the Landolt ring, and it is necessary to be further equipped with a pattern for displaying the test symbol due to a structure of this apparatus, so that it is considered to be difficult to implement technically and operationally. As for a range of the test values, it is described (illustrated) only the test values from 0.1 to 2.0 in the text and FIGS. 5 to 9, and functions for the test values beyond 2.0 and the test value less than 0.1 are not taken into consideration.

Regarding Patent Literature 2, only Landolt rings are randomly displayed, and it is considered to be structurally difficult to add other symbols. Only the test values in increments of 0.1 are taken into consideration.

Patent Literatures 3 and 4 have been devised on the basis of “Manual of Medical Diagnosis of Children and Students” issued from The Japan Society of School Health in 2015, and have been commercialized as the model NKC-370R 8-direction type and 4-direction type disclosed in Non Patent Literature 6. However, only the Landolt ring is used as the test symbol, and only three types of test values of 0.3, 0.7, and 1.0 can be displayed; the test is in such a manner that the judgement is D when a direction of the Landolt ring for 0.3 cannot be recognized, C when a direction of the Landolt ring for 0.7 cannot be recognized, B when a direction of the Landolt ring for 1.0 cannot be recognized, and A when a direction of the Landolt ring for 1.0 can be recognized, and it is not possible to test in further detail by displaying other test values or to display the test symbols such as characters and numbers other than the Landolt ring or test symbol such as a picture supporting infants.

In Non Patent Literature 1, as described in a chart list, the test symbols are prepared in a wide range only for the Landolt ring, but this can be displayed only as a pattern by one chart for each test value, so that each of the Landolt rings in the chart cannot be individually and randomly displayed or cannot be optionally selected and displayed. In addition, as the visual acuity is lowered, the number of types of test symbols that can be displayed gradually decreases to five types and three types, and for 0.09 or less, the visual acuity can be measured only with one test symbol such as “downward Landolt ring” for 0.09, “upward Landolt ring” for 0.08, “rightward Landolt ring” for 0.07, “upward Landolt ring” for 0.06, “downward Landolt ring” for 0.05, “rightward Landolt ring” for 0.04, and “leftward Landolt ring” for 0.03, and no other test symbols such as hiragana characters are not provided, so that it is not possible to perform optometry a plurality of times for confirmation for the same test value, and it is easily memorized when testing the left eye after testing the right eye.

In Non Patent Literature 2, as described in the chart type and specification, the range in which the visual acuity can be measured is from 2.0 to 0.04 in total; there are only 16 types of test symbols for 2.0, 1.5, 1.2, and 1.0 between 1.0 to 2.0, furthermore, for 1.0 or less, there is a test value for which the test cannot be performed because there is no test symbol for 0.09, 0.07, and 0.05, for example, the number of types of the test symbols for each test value gradually decreases from three types or so, and for the test value of 0.04, the visual acuity can be measured with only one test symbol such as “to” for type A and “ko” for type D regarding hiragana characters, “3” for type A and “8” for type D regarding numbers, “E” for type A and “K” for type D regarding alphabets, “upward” for type A and “rightward” for type D regarding the Landolt ring, so that it is not possible to perform optometry a plurality of times for confirmation for the same test value, and even when a plurality of test symbols such as hiragana characters, numbers, alphabets, and Landolt rings is used, it is easily memorized when testing the right eye after testing the left eye. Theoretically, it is necessary that all the provided characters and symbols such as 50 characters of hiragana characters, 26 characters of alphabets, and 8 directions of Landolt ring can be used for all the test values, but it is not possible to randomly display the test symbols for all the test values, it is not possible to individually and randomly display each of the Landolt rings in the chart, or it is not possible to optionally select and display.

In Non Patent Literature 1, only a test value range from 2.0 to 0.1 is assumed, and as described in the chart list, the test symbols are provided in a wide range only for the Landolt ring, but the visual acuity can be measured only for 2.0, 1.5, 1.2, 1.0 to 0.2, 0.15, 0.1, and 0.09 to 0.03.

In Non Patent Literature 2, as described in the chart type and specification, the visual acuity can be measured only in a range from 2.0 to 0.04 as a whole, and it is possible to measure only for the test values similar to those in Non Patent Literature 1 from 2.0 to 1.0 within the range, and for the test value of less than 1.0, the test values of 0.09, 0.07, and 0.05 are not provided, and the test cannot be performed for those test values.

In the test method by the pattern system in Patent Literature 1 or the chart system in Non Patent Literatures 1, 2, 4, and 5, if the test value is to be displayed steplessly or the test symbol is to be randomly displayed, it is necessary to be equipped with all the patterns or the charts of all the test symbols in an overlapping manner for each test value, and accordingly, the data becomes enormous and the operation method becomes complicated, which requires much labor and operation time.

Non Patent Literature 3 is intended to be used by an individual as a guide, and when visiting an eye specialist, purchasing a spectacle or a contact lens, or undergoing optometry at an examination organization such as a vehicle license examination center, approval by the test at that place is required. In addition, only the Landolt ring is provided as the test symbol, so that the test is not accurate.

In the test devices used for acquisition of the vehicle license or the like as in Non Patent Literatures 4 and 5, the pass requirement is 0.7 or more for a normal vehicle license or 0.8 or more for a large vehicle license with both eyes, and the test symbol display of 0.9 or more or less than 0.1 is not required, so that the range of the test values is also equipped only in increments of 0.1 from 0.9 to 0.1. The test symbol is only a Landolt ring, and only two types of leftward and downward rings are provided for 0.1. Since the test symbol charts of the same size for each test value are provided in an overlapping manner in a simple specification, a detailed test cannot be performed.

Model LCD-7000E disclosed in Non Patent Literature 6 is one of the latest visual acuity test devices available from this company, and only 13 steps of test values are set in increments of 0.1 from 0.1 to 1.0 and 1.2, 1.5, and 2.0, so that in a case of a subject with visual acuity of 0.1 or less, it is necessary to shorten the distance to the optometry device or perform the test using other tools such as a handheld test symbol card. Only the Landolt ring can be displayed as the test symbol, and it is not possible to display the test symbols such as the characters or numbers other than the Landolt ring or the test symbol such as a

picture supporting infants, similarly to paragraph described above.

In addition, examples of test instruments such as the model NKO-500, models NB-K and NB-C, models NH-R and NH-L, model NH-I, model HP-1258, three-page visual acuity chart card, model NH-3, model HP-1263, dot card (rabbit and bear) of MORIZANE, model ST Janken card, and model HP-1268 disclosed in Non Patent Literature 6 are available from various companies; however, there is no visual acuity test apparatus that can cover with one instrument at present, and this indicates a current state in which the test must be performed using various test instruments in combination.

Regarding Non Patent Literature 7, in a case of the CA-1000, the test values are in 12 steps from 0.1 to 1.5 with the Landolt ring in one direction for each test value; and in a case of the CA-2000, four steps of 0.02, 0.04, 0.05, and 0.07 are added, but the Landolt ring is only in one direction for each test value, so that it is not possible to test for the same test value with the Landolt rings in a plurality of directions. Only the Landolt ring can be displayed as the test symbol, and it is not possible to display a test symbol of characters or numbers other than the Landolt ring or a test symbol such as a picture supporting infants, similarly to the paragraph described above.

Non Patent Literatures 1, 2, 4, 5, and 7 are all expensive apparatuses, and as described in the paragraphs [0005] to [0009], [0011], and [0014], these devices cannot set the test value steplessly, and the test symbol cannot be randomly selected for all the test values.

### CITATION LIST

### Patent Literature

Patent Literature 1: JP 2007-143665 A

Patent Literature 2: JP H05-154105 A

Patent Literature 3: JP 3010759 Y

Patent Literature 4: JP 3006934 Y

### Non Patent Literature

Non Patent Literature 1: “MIRACLE CHART MC-5/MC-5S”, Topcon Corporation

Non Patent Literature 2: “Liquid Crystal Optotype System Chart SC-1600Pola™/SC-1600”, NIDEK CO., LTD.

Non Patent Literature 3: “Easy Vision Exam (Tablets are also supported)”, Barbaroi Ware

Non Patent Literature 4: “Kowa AS-27x”, Kowa Company, Ltd.

Non Patent Literature 5: “Kowa AS-7H”, Kowa Company, Ltd.

Non Patent Literature 6: “Nitto Insurance Catalog Vol. 337, 2020-2021” NITTO KAGAKU CO., Ltd, model LCD-7000E (pp. 030 to 031), model NKO-500 (pp. 032 to 033), model NKC-370R 8-direction type and 4-direction type (p. 034), models NB-K and NB-C, models NH-R and NH-L, model NH-I (p. 036), model HP-1258, three-page visual acuity chart card, model NH-3 (p. 037), model HP-1263, dot card (rabbit and bear) of MORIZANE, ST Janken card, and model HP-1268 (p. 038). Non Patent Literature 7: “YAGAMI INC CA-1000, CA-2000”, YAGAMI INC

Non Patent Literature 8: “JIS T 7309:2002”, Japanese Standards Association

Non Patent Literature 9: “visual acuity”, Free Encyclopedia “Wikipedia”

### SUMMARY OF INVENTION

### Technical Problem

In a case of examining a visual acuity in a medical institution or the like, the test is performed using various precision test devices equipped with a computer technology, but in most cases, a visual acuity chart in which Landolt rings or characters of each country arranged in order in accordance with test values are displayed on a paper medium or an electric bulletin board is used. This is because, as described in the paragraphs to [0009], [0011], and above, these precision test devices cannot set the test value steplessly, and there is a structural problem that it is not possible to randomly or optionally select all the test symbols such as characters and numbers other than the Landolt rings and pictures for infants, and it is not possible to support all subjects.

The visual acuity chart displayed on the paper medium or the electric bulletin board makes it possible to perform optometry with various types of test symbols such as the Landolt rings, characters, and numbers, but since the number of test symbols that can be displayed is limited, when measuring the visual acuities of the right and left eyes, the symbols are already memorized when the visual acuity of the right eye is measured after the left eye. In particular, as for the symbol for measuring 0.1, there usually are no more than three Landolt rings or so at the top. This leads to a dilemma or frustration that the subject replies “I cannot see.” even though the subject has already memorized and knows the answer, or replies truthfully what the subject sees based on the reason that the subject does not want to lie about what the subject knows. In particular, this might often occur with young children. Since many test symbols have to be displayed, only up to 0.1 can be displayed, and in a case where it is necessary to measure less than 0.1, it is necessary to measure while shortening a distance from the test chart to the subject for the uppermost symbols such as the Landolt rings already known, or it is necessary for the examiner to measure while changing the direction of the Landolt ring upward, downward, leftward, rightward, or diagonally, or changing the test symbols such as the characters, numbers, and pictures while carrying a test symbol card in the hand and shortening the distance to the subject.

Today, there are applications for tablets and smartphones that enable individuals to test; however, they have disadvantages such as difficulty in keeping a constant distance, ambiguous brightness, a function of only the Landolt ring, and a short distance between the subject and the display screen and a limited size of the display screen, leading to difficulty in displaying a large test symbol for 0.1 or less, so that the development has not been made so far, and this is merely a test for reference by him/herself. In fact, when considering why it is necessary to measure the visual acuity, this is for diagnosis of eye disease, purchase of a spectacle or a contact lens, physical checkup, acquisition of various licenses and qualification and the like; it is not possible to adopt the visual acuity measured by oneself, and it is formally essential that an examiner who is a staff belonging to each institution measures the visual acuity of the subject.

In a spectacle shop or the like, a computer-controlled test device is used; however, as described in the paragraphs [0004] to [0009] above, the display of the test value and the test symbol is limited, and the device is expensive, so that the test device cannot be purchased in various facilities such as schools where the use frequency is low.

From the description above, a test apparatus in which a test operation instrument and a test symbol display screen are integrated that enables stepless setting of the test values when performing a visual acuity test in each examination organization, and enables optometry based on a plurality of test results without dilemma or stress of the subject by completely randomly or optionally displaying all the test symbols of various types for all the test values.

### Solution to Problem

A test apparatus according to the present invention includes a visual acuity test operation instrument including an input unit for inputting an optional test symbol by computer control, a storage unit that stores input test symbol data, a test symbol selection unit capable of randomly or optionally selecting the test symbol, and a test value selection unit capable of steplessly selecting a test value; and a test symbol display instrument that displays information such as a test setting and a test symbol processed by the visual acuity test operation instrument.

According to the present invention, the test symbol displayed on the test symbol display screen can be set to one, and a space can be kept smaller than an area of the conventional visual acuity chart using the paper medium or the electric bulletin board.

According to the present invention, a computer program is individually provided with various test symbols such as the Landolt rings in eight directions of upward, downward, rightward, leftward, and oblique directions, 50 hiragana characters, 26 alphabets, characters of other countries, numbers, and pictures, so that even in a case where a plurality of test symbols is displayed on one screen, each of them can be randomly changed independently.

The present invention makes it possible to display the test symbol in a stepless size by enlarging or reducing the test symbol by comparison in accordance with the test value according to criteria disclosed in Non Patent Literatures 8 and 9.

The present invention eliminates a limited test method in which it is possible to test with only three directions of the Landolt rings and only three hiragana characters “ha”, “he”, and “to” for 0.5, with only two directions of the Landolt rings and only two hiragana characters “fu” and “to” for 0.04, for example, and enables display of the Landolt rings in upward, downward, rightward, leftward, and diagonal directions, hiragana characters, katakana characters, Chinese characters, alphabets, languages of other countries, numbers, pictures, and other test symbols wanted to be displayed.

In the present invention, it is possible to completely randomly display the test symbols by a computer program without using any pattern in the test method, or an examiner can optionally select and display the test symbol. In Japanese, characters such as “wo” and “n” are not used alone, so that it is possible to optionally make setting while eliminating such characters in advance.

In the present invention, it is possible to designate only the required test symbols and randomly display for all the test values.

The present invention makes it possible to intentionally display all types of test symbols provided for all test values.

According to the present invention, it is possible to change, a pattern or a configuration of an image such as an icon displayed on the screen, the type of the test symbol, the character, picture, number, and test value, or increase or decrease the number of test symbols to be displayed at the same time by updating.

A preferred embodiment of the present invention is a visual acuity test apparatus including a visual acuity test operation instrument including an input unit for inputting an optional test symbol by computer control, a storage unit that stores input test symbol data, a test symbol selection unit capable of randomly or optionally selecting the test symbol, and a test value selection unit capable of selecting test values from 5.0 to 2.0 in increments of 1.0 and selecting test values from 2.0 to 0.01 in increments of 0.01, and a test symbol display instrument that displays information such as a test setting and a test symbol processed by the visual acuity test operation instrument.

A preferred embodiment of the present invention is a visual acuity test apparatus including a visual acuity test operation instrument formed of a computer, and a test symbol display instrument that displays a test symbol processed by the visual acuity test operation instrument, in which
the visual acuity test operation instrument includes:
a test symbol setting unit capable of selecting test symbols of an optional type out of a plurality of types of test symbols;
a test symbol selection unit capable of randomly or optionally selecting test symbols to be displayed on the test symbol display instrument out of the test symbols of the type selected by the test symbol setting unit; and
a test value selection unit capable of increasing and decreasing a test value of the test symbols selected by the test symbol selection unit,
executes a process of randomly selecting a test symbol out of the test symbols selected by the test symbol selection unit each time the test value selection unit performs an input operation of enlarging and reducing the test symbol; and
the test symbol display instrument
displays the test symbol randomly selected by the visual acuity test operation instrument.

In a preferred embodiment of the present invention, the visual acuity test operating instrument
executes, when a plurality of test symbols is displayed side by side on the test symbol display instrument, a process of individually and randomly selecting each of the test symbols to be displayed side by side out of the test symbols selected by the test symbol selection unit each time the test value selection unit performs an input operation of enlarging and reducing the test symbol, and
the test symbol display instrument
simultaneously displays each of the test symbols individually and randomly selected by the visual acuity test operation instrument side by side.

In a preferred embodiment of the present invention, the visual acuity test operation instrument increases the number of test symbols individually and randomly selected out of the test symbols selected by the test symbol selection unit by one or more each time the test value exceeds a predetermined test value when increasing the test value stepwise by the test value selection unit, and allows the test symbol display instrument to display, and decreases the number of test symbols individually and randomly selected out of the test symbols selected by the test symbol selection unit by one or more each time the test value becomes lower than a predetermined test value when decreasing the test value stepwise by the test value selection unit, and allows the test symbol display instrument to display.

In a preferred embodiment of the present invention, the visual acuity test operation instrument allows the test symbol display instrument to display a single test symbol randomly selected out of the test symbols selected by the test symbol selection unit when the test value selected by the test value selection unit is 0.01, allows the test symbol display instrument to display two test symbols randomly selected out of the test symbols selected by the test symbol selection unit when the test value is 0.02, allows the test symbol display instrument to display three test symbols randomly selected out of the test symbols selected by the test symbol selection unit when the test value is 0.03 or 0.04, allows the test symbol display instrument to display four test symbols randomly selected out of the test symbols selected by the test symbol selection unit when the test value is in a range from 0.05 to 0.09, and allows the test symbol display instrument to display five test symbols randomly selected out of the test symbols selected by the test symbol selection unit when the test value is in a range from 0.1 to 2.0.

### Advantageous Effects of Invention

A visual acuity test apparatus according to the present invention is a test apparatus assuming a visual acuity test apparatus used at a medical institution, a spectacle shop, and when acquiring various licenses and qualifications. A test symbol is completely randomly or optionally displayed, and it is possible to get a visual acuity test at a constant distance by one test apparatus without necessity that a subject moves back and forth in the test up to 0.01, for example, or an examiner moves back and forth with respect to the subject with another Landolt ring card or the like carried in the hand.

In the present invention, the test symbol is displayed completely randomly or optionally, so that the subject cannot memorize the same and can get the test without feeling dilemma or stress.

In the present invention, the examiner can display the test symbol completely randomly, and the time and effort for selecting them can be eliminated, the test can be smoothly performed, and the time can be shortened. This is more effective when the number of subjects is large.

According to the present invention, it is not necessary to prepare charts and patterns in advance as in the conventional art, and the symbols such as hiragana characters, alphabets, languages of other countries, numbers, and pictures can be displayed for all the test values, and even in a case of displaying a plurality of test symbols on one screen, each of them can be changed independently and randomly.

Since the present invention is an apparatus including only an operation instrument in remote control and a test symbol display screen, the operation instrument in remote control is as large as a keyboard or a tablet terminal. Like a normal keyboard for computer, a screen for displaying an operation status on the remote control operation instrument is not necessarily required, and in a case of a keyboard without screen, a test symbol display instrument can directly display an operation screen. When one test symbol is displayed on the test symbol display screen, in a case where a test symbol for measuring 0.01 from a distance of 5 m is displayed, the size of the symbol is 750 mm in diameter when represented in a Landolt ring by comparison in accordance with the test value according to the standard disclosed in Non Patent Literatures 8 and 9, so that only a size slightly larger than the above size is required as the size of the screen, and the space can be made smaller than the area of the conventional visual acuity chart. In the future, with the progress of technological innovation, one thin sheet excellent in portability is sufficient, and can be used not only in examination facilities such as a medical institution, a spectacle shop, and vehicle license examination center, but also in facilities such as schools and mobile medical care. A smaller display instrument can be used in a case where the test distance is 3 m or 1 m.

According to the present invention, it is possible to test in a cross-sectional manner with the Landolt rings, characters of each country corresponding to the subject, numbers, pictures, and other symbols with one operation for all test values.

Since the present invention uses only a small space, a color perception test, an astigmatism test, and other test functions can also be added.

The test operation instrument and the test symbol display instrument according to the present invention may be connected wirelessly or by wire to be used.

Conventionally, in a case of measuring a visual acuity, 2.0 is usually used as the best visual acuity test value, and the concept is recognized as a matter of course, so that a commercialized visual acuity chart displayed on a paper medium or an electric bulletin board, and various precision test devices equipped with computer technology are made based on the same. However, the best value of 2.0 is not defined as a standard, and unlike such inspection instrument or the like, in the present invention, there is no restriction on the test value and the number of test symbols that can be displayed as described in [0018] and [0019] above, so that it is possible to display the test symbol smaller than before, and measure the visual acuity beyond 2.0. At that time, considering that a difference in size of the test symbol occurs less as the test value increases, it is also conceivable to display 3.0, 4.0, and 5.0 in increments of 1.0 after 2.0. As described in above, the conventional visual acuity test has focused on its efficiency, countermeasures for classifying whether or not a subject uses a straightener such as spectacle or the like, and has not considered measuring the visual acuity up to a higher measured value; however, if it is possible to measure the visual acuity beyond 2.0 by changing the conventional concept, people have more willingness to have a better visual acuity than before, and it may lead to research and development on today's problems that deterioration thereof is concerned due to use of electronic devices or the like.

### BRIEF DESCRIPTION OF DRAWINGS

FIG. 1 is a test setting operation screen of a visual acuity test operation instrument.

FIG. 2 is a Landolt ring symbol operation screen of the visual acuity test operation instrument.

FIG. 3 is a hiragana character symbol operation screen of the visual acuity test operation instrument.

FIG. 4 is an alphabet symbol operation screen of the visual acuity test operation instrument.

FIG. 5 is a color picture symbol, a silhouette picture symbol, and a numerical symbol operation screen of the visual acuity test operation instrument.

FIG. 6 is a Landolt ring test symbol display example of a test symbol display instrument.

FIG. 7 is a hiragana character test symbol display example of the test symbol display instrument.

FIG. 8 is an alphabet ring test symbol display example of the test symbol display instrument.

FIG. 9 is a color picture test symbol display example of the test symbol display instrument.

FIG. 10 is a silhouette picture test symbol display example of the test symbol display instrument.

FIG. 11 is a number test symbol display example of the test symbol display instrument.

FIG. 12 is a drawing of single test symbol display of the test symbol display instrument.

FIG. 13 is a drawing of plural test symbol display of the test symbol display instrument.

FIG. 14 is an example of visual acuity test.

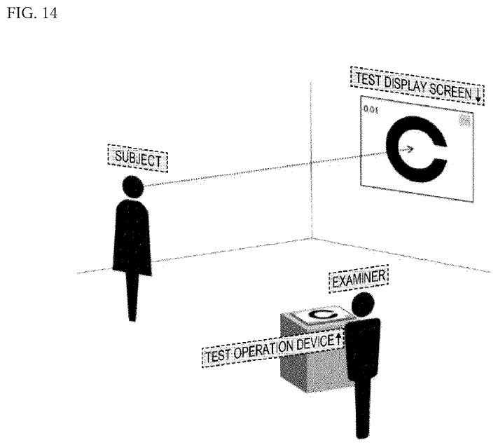

### DESCRIPTION OF EMBODIMENTS

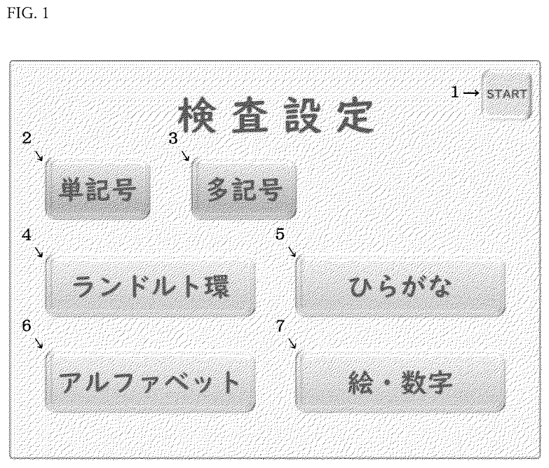

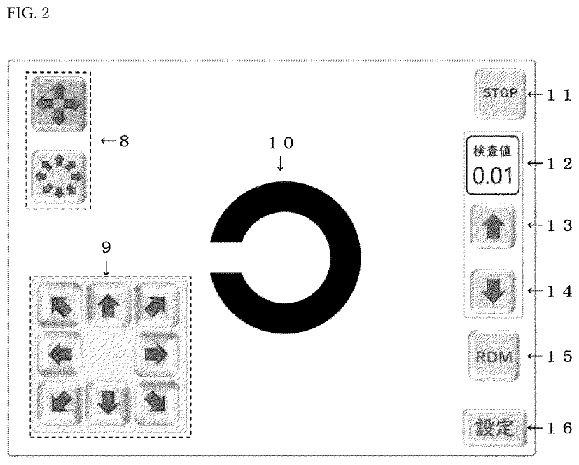

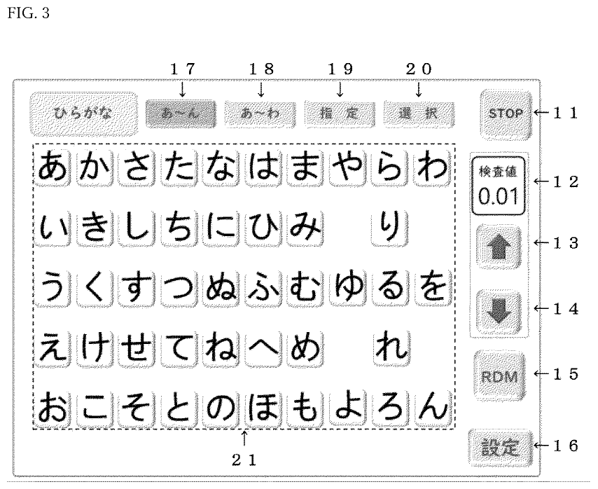

A test is started by clicking reference numeral 1 in FIG. 1 of an input unit for inputting a test symbol, the test is ended by clicking reference numeral 11 in FIG. 2, reference numeral 12 represents a test value, the value can be changed by enlarging or minimizing the test symbol by one step each time reference numeral 13 or 14 is clicked, it moves to FIG. 1 by clicking reference numeral 16, and reference numerals 11, 12, 13, 14, 15, and 16 have the same function on test screens in FIGS. 3, 4, and 5.

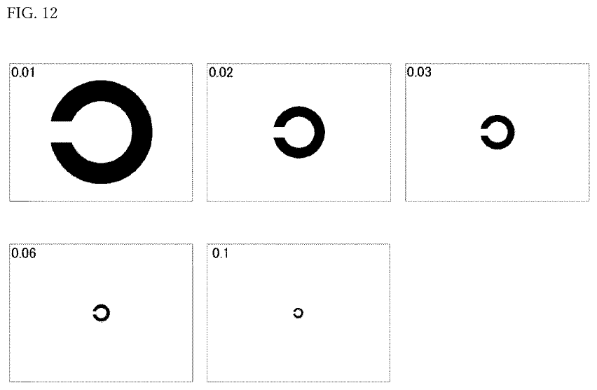

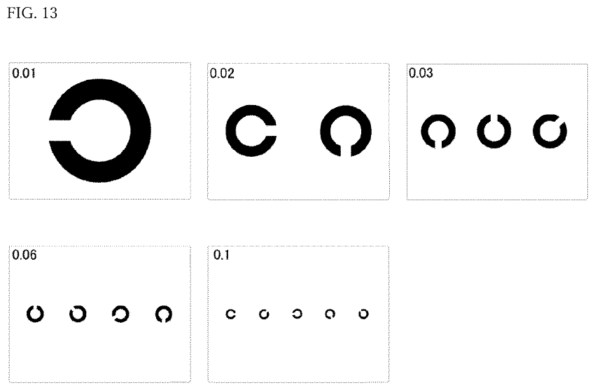

In FIG. 1 of the input unit for inputting the test symbol, when reference numeral 2 is clicked, every test symbol is displayed in single for all the test values as in a screen view illustrated in FIG. 12, and when reference numeral 3 is clicked, every test symbol is displayed in single for 0.01 and displayed in plural for other test values such as two for 0.02, three for 0.03 and 0.04, four for 0.05 to 0.09, and five for 0.1 to 2.0 as in a screen view illustrated in FIG. 13. Each of the test symbols can be individually and randomly displayed not only when this is displayed in single but also when this is displayed in plural.

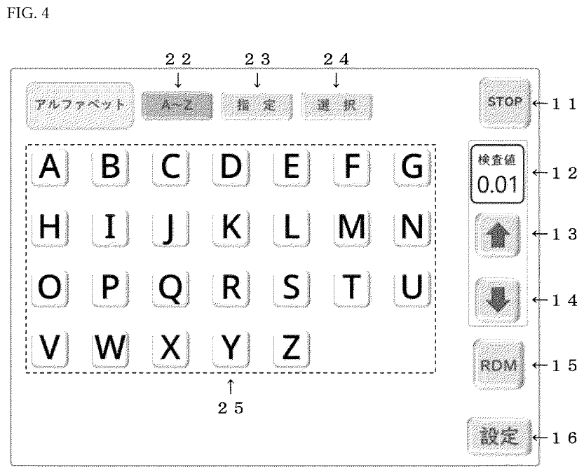

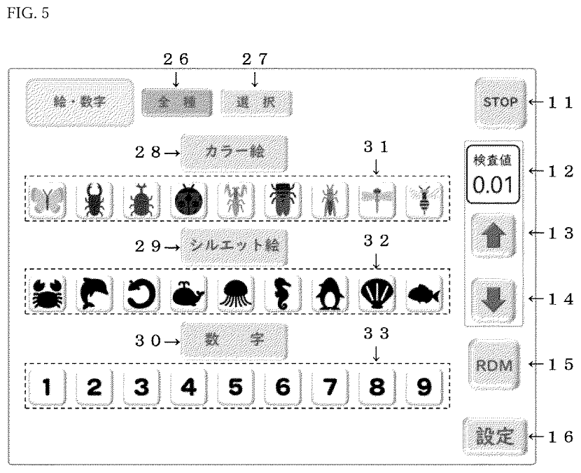

In FIG. 1 of the input unit for inputting the test symbol, when reference numeral 4 is clicked, it moves to an operation screen provided with an input unit, a storage unit, a selection unit, and a test value selection unit for a Landolt ring symbol in FIG. 2, when reference numeral 5 is clicked, it moves to an operation screen provided with an input unit, a storage unit, a selection unit, and a test value selection unit for hiragana character symbols in FIG. 3, when reference numeral 6 is clicked, it moves to an operation screen provided with an input unit, a storage unit, a selection unit, and a test value selection unit for alphabet symbols in FIG. 4, and when reference numeral 7 is clicked, it moves to an operation screen provided with an input unit, a storage unit, a selection unit, and a test value selection unit for picture and number symbols in FIG. 5.

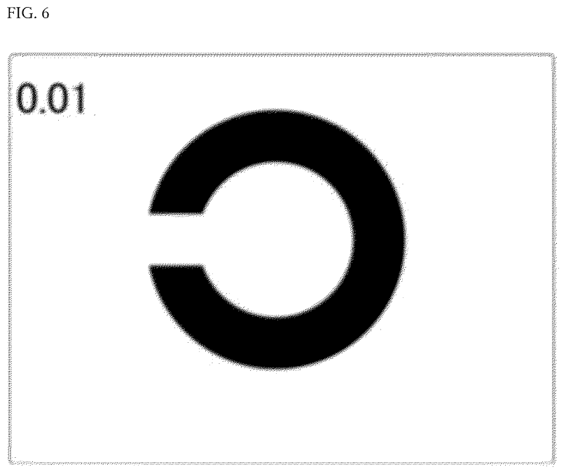

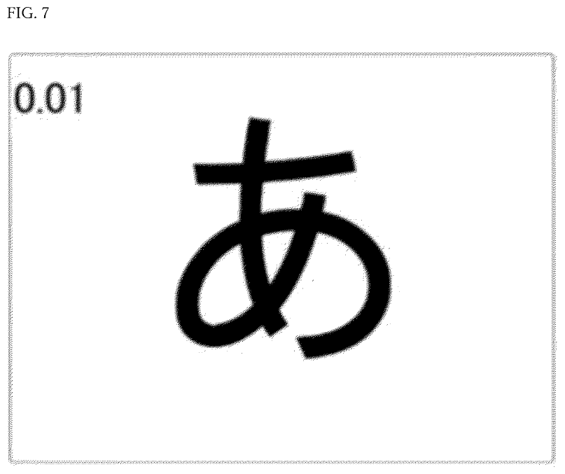

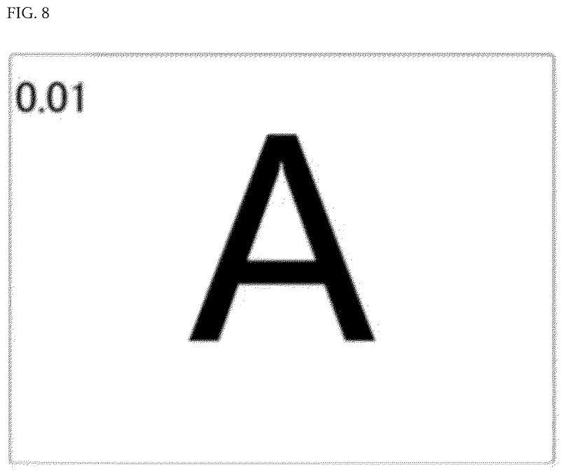

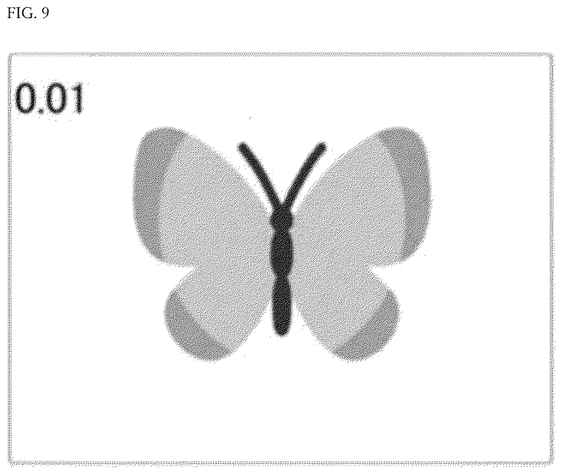

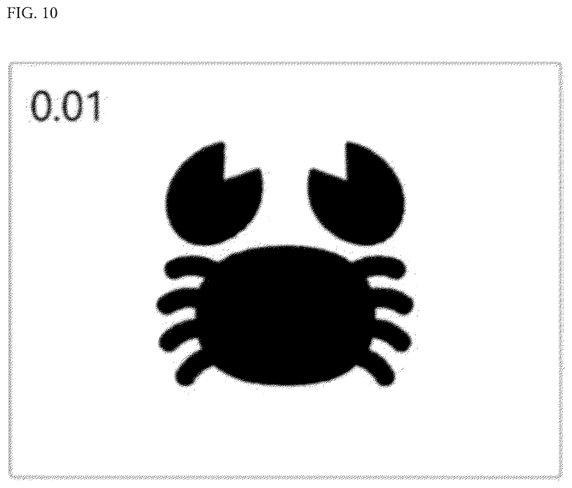

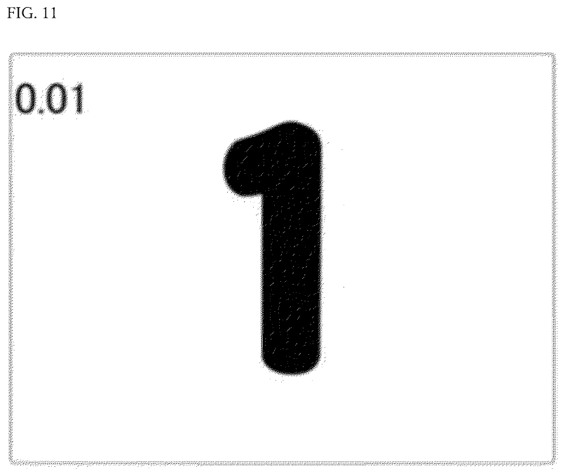

FIGS. 6, 7, 8, 9, 10, and 11 are screen views of a test symbol display instrument with which a subject who views the same undergoes a test, in which FIG. 6 illustrates a test symbol and a test value input, stored, and selected in FIG. 2, FIG. 7 illustrates a test symbol and a test value input, stored, and selected in FIG. 3, FIG. 8 illustrates a test symbol and a test value input, stored, and selected in FIG. 4, FIG. 9 illustrates a test symbol and a test value input, stored, and selected when clicking reference numeral 28 in FIG. 5, FIG. 10 illustrates a test symbol and a test value input, stored, and selected when clicking reference numeral 29 in FIG. 5, and FIG. 11 illustrates a test symbol and a test value input, stored, and selected when clicking reference numeral 30 in FIG. 5.

FIG. 2 of the input unit for inputting the test symbol is a screen view after clicking reference numeral 4 in FIG. 1, in which it is illustrated that 4-direction or 8-direction is selected by clicking any one of two icons represented by reference numeral 8, and every time reference numeral 15 is clicked, a direction of a break of a Landolt ring represented by reference numeral 10 is randomly selected and displayed. Every time any one of reference numeral 9 is optionally clicked, a symbol of the Landolt ring with a break in a direction of the arrow is displayed.

FIG. 3 of the input unit for inputting the test symbol is a screen view after clicking reference numeral 5 in FIG. 1, in which all 50 hiragana characters are selected by clicking reference numeral 17 and they are randomly displayed every time reference numeral 15 is clicked, 48 hiragana characters except for “wo” and “n” are selected by clicking reference numeral 18 and they are randomly displayed every time reference numeral 15 is clicked, a character wanted to be displayed out of reference numeral 21 may be optionally designated by clicking reference numeral 19 and they are randomly displayed every time reference numeral 15 is clicked, and by clicking reference numeral 20, every time any character in reference numeral 21 is clicked, the symbol is displayed.

FIG. 4 of the input unit for inputting the test symbol is a screen view after clicking reference numeral 6 in FIG. 1, in which all 26 alphabets are selected by clicking reference numeral 22 and they are randomly displayed every time reference numeral 15 is clicked, a character wanted to be displayed out of reference numeral 25 may be optionally designated by clicking reference numeral 23 and they are randomly displayed every time reference numeral 15 is clicked, and by clicking reference numeral 24, every time any character in reference numeral 25 is clicked, the symbol is displayed.

FIG. 5 of the input unit for inputting the test symbol is a screen view after clicking reference numeral 7 in FIG. 1, in which color pictures in reference numeral 31 are selected by clicking reference numeral 28, silhouette pictures in reference numeral 32 are selected by clicking reference numeral 29, numbers in reference numeral 33 are selected by clicking reference numeral 30, all the symbols of the selected type are designated by clicking reference numeral 26 and they are randomly displayed every time reference numeral 15 is clicked, and by clicking reference numeral 27, every time any symbol of the selected type of any of reference numeral 31, 32, or 33 is clicked, the symbol is displayed.

The test value selection unit that selects the test value steplessly can display the test symbol in a stepless size by enlarging or reducing the test symbol by comparison in accordance with the test value according to the criteria disclosed in Non Patent Literatures 8 and 9; in this test apparatus, it is possible to display in increments of 1.0 from 5.0 to 2.0, display in increments of 0.1 from 2.0 to 1.0, and display in increments of 0.01 from less than 0.1 to 0.01 to test, but it is also possible to display the test value beyond 5.0, and 0.32, 0.15 or the like.

Every time each of all the icons is clicked, a color of the icon changes to indicate that the icon is selected.

It is possible to change the type of the test symbol, the character, picture, number, and test value, or increase or decrease the number of test symbols to be displayed at the same time by updating.

### Reference Signs List

1 Test start icon
2 Test symbol single display icon
3 Test symbol plural display icon
4 Landolt ring setting icon
5 Hiragana character setting icon
6 Alphabet setting icon
7 Picture/number selection icon
8 Landolt ring 4-direction/8-direction selection icon
9 Landolt ring direction designation icon
10 Display for confirming direction of Landolt ring
11 Test end icon
12 Test value display
13 Test symbol enlarged display icon (enlarged by one step every time clicked)
14 Test symbol reduced display icon (reduced by one step every time clicked)
15 Test symbol random selection icon
16 Test setting screen display icon
17 All-hiragana character selection icon
18 48-hiragana characters (except for “wo” and “n”) selection icon
19 Hiragana character designation icon
20 One-hiragana character selection icon
21 Hiragana character icon
22 All-alphabet selection icon
23 Alphabet designation icon
24 One-alphabet selection icon
25 Alphabet icon
26 All-color picture or silhouette picture or number selection icon
27 One-symbol selection icon out of color picture or silhouette picture or number
28 Color picture setting icon
29 Silhouette picture setting icon
30 Number setting icon
31 Color picture icon
32 Silhouette picture icon
33 Number icon

1. A visual acuity test apparatus comprising: a visual acuity test operation instrument including an input unit for inputting an optional test symbol by computer control, a storage unit that stores input test symbol data, a test symbol selection unit capable of randomly or optionally selecting the test symbol, and a test value selection unit capable of selecting test values from 5.0 to 2.0 in increments of 1.0 and selecting test values from 2.0 to 0.01 in increments of 0.01; and a test symbol display instrument that displays information such as a test setting and a test symbol processed by the visual acuity test operation instrument.
